(19) United States
(12) Patent Application Publication
(10) Pub. No.: US 2019/0184202 A1
(43) Pub. Date: Jun. 20, 2019

(54) Hand-held Battery-Operated Therapeutic Ultrasonic Device

(71) Applicants: Gholam Hossein ZERESHKIAN, Richmond Hill (CA); Jahangir TAVAKKOLI, Richmond Hill (CA); Kevin ROD, Toronto (CA)

(72) Inventors: Gholam Hossein ZERESHKIAN, Richmond Hill (CA); Jahangir TAVAKKOLI, Richmond Hill (CA); Kevin ROD, Toronto (CA)

(21) Appl. No.: 15/843,898

(22) Filed: Dec. 15, 2017

(51) Int. Cl. A61N 7/00; B06B 1/06; B06B 1/02

(52) U.S. Cl. CPC A61N 7/00; B06B 1/06; A61N 2007/0021; B06B 1/0253; A61N 2007/0034; B06B 1/023

(57) ABSTRACT

The present invention is a portable hand-held battery-operated therapeutic ultrasonic device that is specifically designed for clinical applications in muscle and joint pain management. The present invention generates stepwise high resolution, high frequency microprocessor-based signal to drive piezo-crystals for applying on the skin of the user. It makes use of a novel electronic driving technology that enables it to generate therapeutically combinations of acoustic intensities from 0 to 2 W/cm² and with 80-85% efficiency. It is a lightweight device and can be programmed wirelessly by a physician/professional to be used at home by a patient.

### FIELD OF THE INVENTION

The present invention relates in general to Medical Devices, and especially to Pain Management and Skin Treatment Devices.

### BACKGROUND OF THE INVENTION

The pain treatment in muscles and joints are a common issue for general public and more importantly for the elderly population. Typical treatments usually require a number of visits to pain specialist physicians and/or physiotherapists leading to a significant burden on health care systems, insurance companies and governments. One of the most successful technologies being used for muscles and joints pain management is therapeutic ultrasound, which typically makes use of ultrasonic waves with a frequency range of 0.5-5 MHz and with ultrasonic intensities of up to 2 W/cm². All ultrasonic therapeutic devices that are currently commercially available in the market for muscle/joint pain management are AC power operated and office based with price tags of at least a few thousand dollars. Currently available devices that could generate the range of output acoustic powers (or intensities) required for ultrasonic physical therapy, i.e., up to 2 W/cm², are all office based and bench topped. They are neither hand-held nor battery-operated.

A muscle/joint pain management ultrasonic device that is hand held, battery operated and inexpensive will open up a number of non-office based applications of the technology, e.g., in home and in the field.

There are many hand-held portable ultrasonic devices in the market with profiles similar to the present invention. However, they all produce very low level of ultrasonic power, which makes them ineffective in the area of muscle/joint pain management. These devices cannot generate more power due to the way the high frequency signal is generated. They use typical LC oscillators in which a piezo ceramic crystal is a part of the circuit, and needs a high-power transistor to drive and to generate high power ultrasonic energy. This driving electronic design either needs high levels of voltages above the safety range and /or large heat sinks with large cooling capacities, which makes it bulky, heavy, and expensive.

Moreover, all these devices, including the office based ones; lack a reliable warning mechanism to let the user know if the coupling between the transducer surface and the skin is good and if the ultrasonic energy is being effectively transferred to the tissue without any discernible risk of skin burn or damage.

### SUMMARY OF THE INVENTION

The present invention is a portable and handheld battery-operated ultrasonic device that generates stepwise high resolution, high frequency microprocessor- based signal to drive piezoelectric-crystals for applying on the skin of the user. This stepwise signal enables to reduce the power loss in driving circuit leading to increase in the efficiency and allowing the device to operate even with a battery.

In addition, monitoring the amount of power delivered to a piezo-crystal enables the user to determine if there is a proper coupling, e.g., if it is in the air or if it is not delivering an appropriate energy to a tissue.

The present invention generates all combinations of acoustic intensities from 0 W/cm² to 2 W/cm² and with 80-85% efficiency (Piezo mechanical force/input electrical power). It is a lightweight device weighing less than 150 g without battery and less than 350 g with a battery. Pain specialist physicians, physical therapy and physiotherapy practitioners, dermatologists, sport medicine specialists, athletics, beauty salons, general people, and elderly people, can easily use the present device.

The present invention is able to auto tune itself with a piezo-crystal head and is able to detect if the power is not delivered to the tissue and can be programmed wirelessly by a physician/professional to be used at home or by a patient and to reduce the time and cost for them. The versatile electronic design along the capability of handling multi-head piezo-crystals enables it to be used for skin care as well.

One objective of the present invention is to provide a new technology to produce a high power hand-held ultrasonic device having therapeutically effective output, and being programmable to be used under physician supervision while at home.

Another object of the present invention is to provide a device with a capability of handling multi-head piezo-crystals, enabling it to be used for skin care and other applications.

Another object of the present invention is to provide a stepwise shape ultrasound signal with nanosecond pattern enabling to generate a semi Sinus wave form to drive the Piezo-crystal.

Another object of the present invention is to provide a device that can track its operation history, and to determine if the patient has used the device properly.

Another object of the present invention is to resolve the prior art issues, relating to portability, low weight and being battery operated, which allows the present device to be used in places which other devices cannot be used, like in the fields.

Another object of the present invention is to provide a programmable device to program the power, pulse rate and time of operation, enabling physician to track the treatment process and gradually apply the amount of power needed.

Another object of the present invention is to provide a significantly less expensive device as compared with devices currently available in the market with similar specs.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1A shows a front sectional view of a portable hand-held battery-operated therapeutic ultrasonic device according to the present invention;

FIG. 1B shows a perspective view of the present invention;

FIG. 1C shows a perspective view of the present invention with a base;

FIG. 2 is a block diagram illustrating the main elements of the present invention;

FIG. 3 is a block diagram illustrating the driver element according to the present invention, and

FIG. 4 is a block diagram of the processing unit of the present invention.

### DETAILED DESCRIPTION OF PREFERRED EMBODIMENTS

The figures are not intended to be exhaustive or to limit the present invention to the precise form disclosed. It should be understood that the invention can be practiced with modification and alteration, and that the disclosed technology be limited only by the claims and equivalents thereof.

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

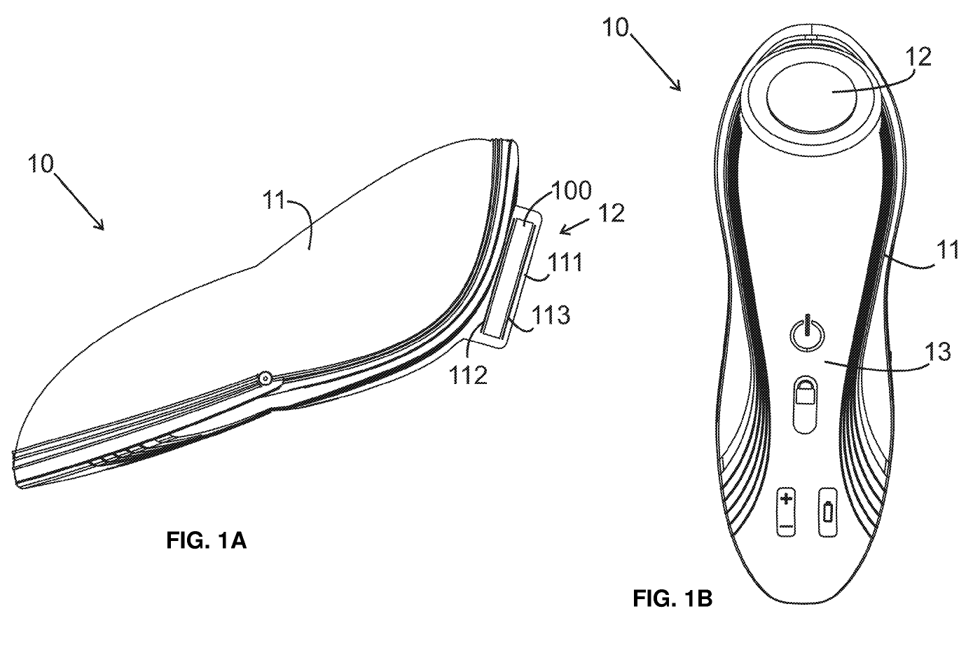

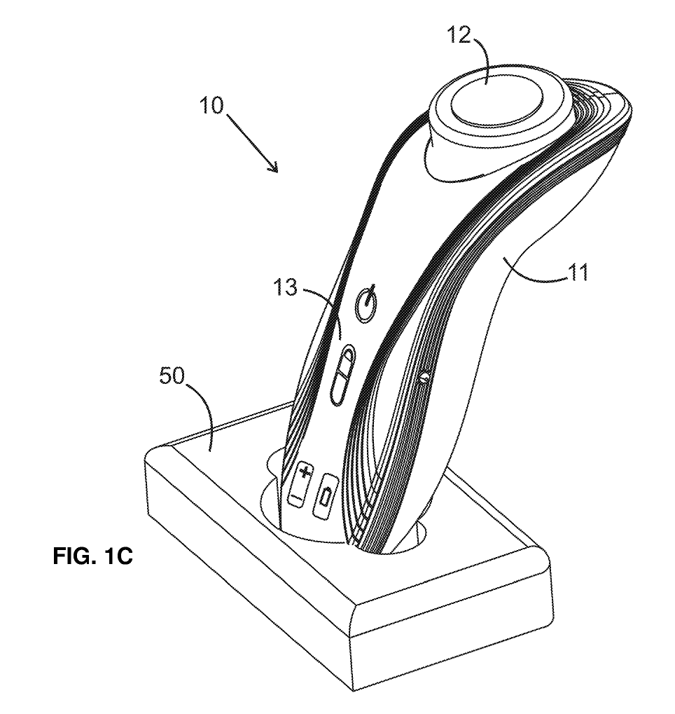

FIGS. 1A, 1B and 1C illustrate a portable and hand-held battery-operated therapeutic ultrasonic device 10 in accordance with a preferred embodiment of the present invention. The device includes a hand-held grip housing 11 provided at its one end thereof with an applicator head 12, which is adapted in use to contact with a user's skin 120 for applying ultrasound thereon. The applicator head 12 is comprised of piezo-crystals 100 generating the ultrasound, and a transmitter 111 transmitting the ultrasound to the skin 120. The piezo-crystals 100 are preferably shaped into a circular disc (but any other shape is possible) having an upper surface and a lower surface, which are covered with upper and lower electrodes 112 and 113 across which an electric pulse is applied for generating the ultrasound vibration.

The piezo-crystals 100 and the transmitter 111 are integrated into a combined vibration mass, which is caused by the electric pulse to resonate for generating and applying the resonant ultrasound vibration to the skin 120. Preferably the device 10 is designed to generate the ultrasound having a wide range of operation from 100 KHz to 4 MHz with 1 KHz Resolution in MHz and its automatic frequency matching technique makes it very Power efficient 80-85% acoustic to electric power ratio. The device 10 further comprises of a display unit 13 to display a range of information thereon according to the present invention.

The present invention may provide a charging station 50 for charging a portable and hand-held battery-operated therapeutic ultrasonic device 10 wherein the charging station 50 is configured for a hand-held part of the device such that the hand-held part has a substantially vertical alignment when charging.

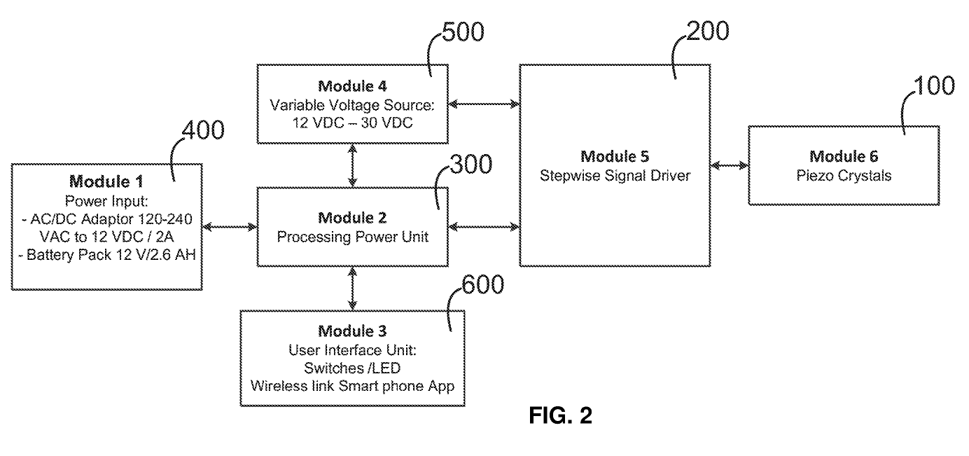

The main elements of the present invention are provided in FIG. 2. The source of the ultrasound vibration is the piezo-crystal 100, which is driven by a stepwise signal driver 200. A processor 300, which itself is powered by an input power source 400, and a variable voltage source 500, which controls the stepwise signal driver 200. A user interface unit 600 controls the processor. The stepwise signal driver 200 reduces the power loss in driving circuit leading to increase in the efficiency, and enabling the device to operate with a battery. The piezo- crystal 100 has a temperature sensor and coded-bits to receive power from the driver and deliver the temperature of piezo-crystal as well as its type for identification to the processing unit 300.

The driver 200 generates stepwise signals as well as measuring the power of the delivered signal to the piezo-crystals 100 and reports it to the processing unit 300.

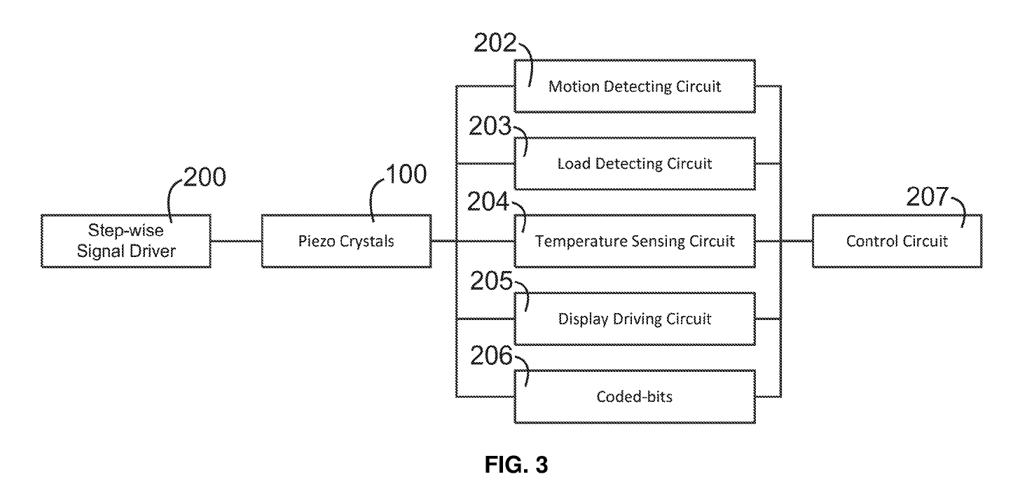

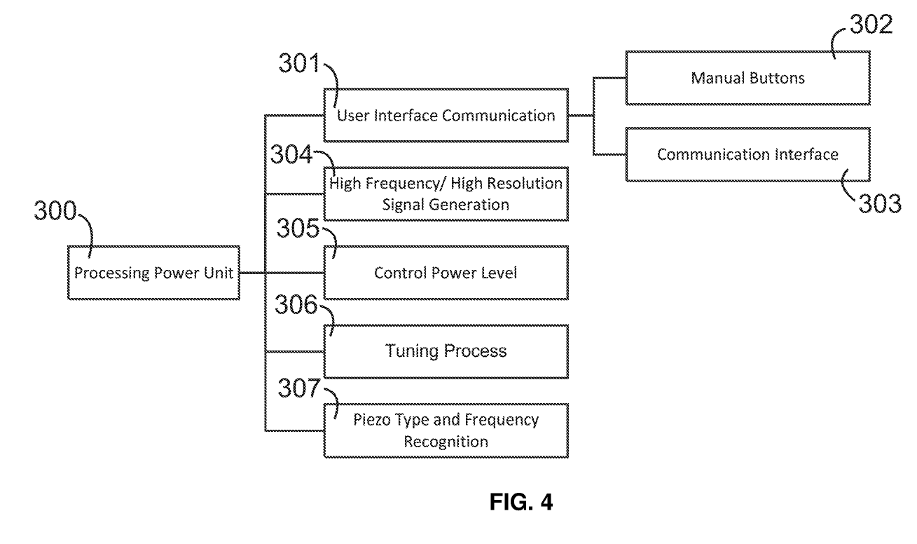

A bridge amplifier is designed to provide a stepwise signal with nanosecond pattern, enabling the device to generate a semi Sinus waveform to drive the piezo-crystals 100. One embodiment of the preferred circuit is shown in FIG. 3 with an output signal depicted in FIG. 4. Four MOSFET switches (SW1-SW4) control the delivery of a suitable signal to the piezo-crystal. In each resonance cycle (the magnified part of which is shown in FIG. 4) “c” represents the period of a signal, which comprises of “a” and “b” intervals. These intervals are automatically set in a way to have an efficient switching delivery of the power to the crystal, compensating switching delays and helping to set the desired power. Since, the resonance frequency changes with load, heat, age and the type of the crystal, it has to be optimized for efficient operation. The resonance frequency is originally set to a nominal frequency, but it is continuously monitored and optimized during the operation. This is done by continuous measurement of the current passing through the piezo, which is sent to the control circuit for compensation.

Each Piezo Crystal on separate head has a built in code which CPU can recognize the Typical Resonance frequency of the used Piezo (for example 400 Khz, 1 Mhz or 3 Mhz Piezo has different code). Resonance Frequency is set by changing the “c” by CPU and with getting feedback from voltage dropped across “R”. The voltage pattern is captured by bursting mani Piezo frequency with low burst signal (like 100 Hz), get the pick and form factor of the demodulated signal and transfer it through isolating amplifier to CPU. Then the CPU searches around the typical resonance frequency by resolution of around 0.2% and determine the actual resonance frequency which can be saved and be used later whenever the device is being on to check again.

Each piezo-crystal has a built in code on its head, which the CPU of the system can recognize. The code contains information on the resonance frequency of the piezo (for example, it can be 400 Khz, 1 Mhz or 3 Mhz). The resonance frequency is read by the CPU and the parameter “c” is adjusted to generate the initial resonance frequency. In operation, when a load is applied on the piezo, the CPU reads the voltage drop across “R” in FIG. 3. Then a search is performed around the initial resonance frequency by a predefined resolution, preferably of around 0.2%, to find the actual resonance frequency. The actual resonance frequency is when the voltage across R is maximum for a given input power. This information is saved in the system. In time, the system learns the optimum conditions for any given load and it quickly adjust accordingly. This significantly increases the device efficiency and effeteness, and reduces power consumption.

In addition, the signal is also optimized for more efficient operation. During the treatment, by measuring the feedback voltage across “R”, the ratio of “a” and “b” can be tuned by the CPU by changing the status of the switches. The waveform, which is like a staircase signal, is generated with the following pattern of switches: Step1: All switches are OFF. Step2: SW1 and SW4 are ON. Step3: All switches are OFF. Step4: SW2 and SW3 are ON. This switch pattern protects the switches and prevents two of them to stay ON simultaneously, thus preventing switch failure. It also can optimize the shape of the waveform and energy applied to the piezo.

The optimum resonance frequency is continuously determined and applied to the piezo-crystal, however, by controlling the amount of power, the bursts can be achieved at lower frequency signals. Parameters of this signal like the voltage level, the make and the brake interval can be set to apply desired power to piezo crystal.

The frequency can continuously be applied to the piezo-crystal. However, in the current method, by applying signal bursts, a low frequency signal (like 1 Khz) but very short duration can be applied (see FIG. 4). Parameters of this signal like the voltage level (for example from 24V p-p to 64V p-p) and duration of the burst signal, which can be from 1% to 100%, as well as the total time of treatment (like 3 minutes) can be set by a practitioner, either manually or by a Bluetooth or wireless link.

According to FIGS. 1A, 1B, 1C and FIG. 5, the stepwise signal driver 200 provides the electric pulse across the electrodes 112 and 113 of the piezo-crystals 100. The driver includes a motion detecting circuit 202 for detection of a motion of the applicator head 12, a load detecting circuit 203 for detecting the load condition of the applicator head 12, a temperature sensing circuit 204 for sensing the temperature of the piezo-crystals 100, a display driver circuit 205 for displaying the operating conditions of the device, a coded-bits unit 206 to determine the type of the piezo-crystal, and a control circuit 207 for control of the above circuits. The driver 200 is energized by a power supply. The device 10 monitors the amount of power delivered to the piezo-crystals and reports it to the processor thereby the appropriate energy delivered to the tissues.

The device 10 is designed to generate the ultrasound while the applicator head 12 is in contact with the skin 120.The load detecting circuit 203 detects whether a suitable load is applied to the skin and determines whether the transmitter 111 is loaded or not and restricts the generation of the ultrasound. The motion detecting circuit 202 is provided to enable the continuous ultrasound application when the applicator head 12 is moving at a suitable rate and otherwise disable or limit the ultrasound generation. This prevents the potential of hazard of causing a cold burn in the skin. In addition, the control circuit 207 includes a timer, which stops generating the ultrasound after the device is utilized over a preset time. The timer operates to continue generating the ultrasound over the preset time. In addition, after the preset time is elapsed, the control circuit 207 gives an instruction to stop providing the electric power to the driver 200, stopping the ultrasound generation.

According to FIG. 6 the processor 300 comprises of a microcontroller based system, which has the following functions:
(i) A user interface communication 301, which has (a) manual buttons (On/Off, power and duty cycle level, visual interface (LEDs) 302, and (b) a wireless communication interface 303 enabling this unit to be used for Cloud Control or remote Control of the device either reduce the length of treatment and/or need for patient to be in Dr's office.
(ii) A high-frequency/high-resolution signal generation 304. Wide range of operation from 100 KHz to 4 MHz with 1 KHz Resolution in MHz operation makes the present invention 10 very precise and its automatic frequency matching technique makes it very Power efficient 80-85% acoustic to electric power ratio.
(iii) A control power level 305 by controlling step up switching power supply. Wide range of voltages from 12V to 35 VDC enables having many combinations of output powers.
(iv) An automatic control 306 for the piezo frequency tuning process by measuring the output current changes in low modulation Frequency with high-resolution frequency checks. The method is able to see the pattern of the best fitting resonance frequency and correct it if needed.
(v) A piezoelectric type and frequency recognition device 307. Each Piezo head has a unique code based on which is able to set the gross tuning frequency. By using the above technique, the fine tune frequency can be set.

Referring to FIG. 2 again, the input power source 400 is responsible to power the device. It can be a universal voltage adaptor 110 Vac-240 Vac to 12 VDC/2 A, Medical device category, or a Li-ion Battery Pack 12.6V/2.4 A. This means that the unit can either work with safe voltage adaptor or safe Battery pack for at least 2 hours. Long life battery operation with a maximum standard acoustic power (2 W/Cm2) and low weight (350 Grams) is a unique feature that makes the present device a real handheld device. The battery pack can be integrated into a housing or multiple interconnected housings. Various components needing to operate or recharge the power are also provided.

The variable voltage or the switching power supply 500 is responsible to deliver the necessary voltage/power to the driver stage based on the request from the processor 300. Switching power supply is controlled by the main micro controller in to enable the system to generate any pattern, so for all scientific experiments can be implemented with the present device.

The user interface 600 comprises of switches and displays as well as a communication link between the device and external application on a smart phones/computers/Cloud. Communication link enables practitioner to set the power, period of use, record the usage by the patient, and check the usage. The device has wireless communication interface to wirelessly communicate with any external processor and computers.

In operation, after turning on a power switch, the stepwise signal driver 200 actuates the piezo-crystals 100 to start vibrating and generating the ultrasound. At this time, the temperature-sensing unit 204 starts sensing. The motion detection 202 and the load detection 203 operate in combination with each other based on the instruction given to the timer.

The applicator head 12 comprises of the piezo- crystals 100 and a transmitter 111. The piezo-crystals 100 are made of a ceramic and are preferably shaped into circular disks having a thickness. An upper electrode 112 and a lower electrode 113 is provided. The transmitter 111 is further shaped into preferably a circular disk having a uniform thickness. The electric pulse from the step-wise driver 200 is applied across the electrodes 112 and 113 and transmitted by the transmitter 111. The piezo-crystal 100 is secured to the transmitter 111 such that it is integrated into a combined vibration mass, which resonates with the electric pulse from the step-wise driver 200 to generate the ultrasound to be transmitted to the skin. The ultrasound effectively transmits to the user's skin.

Another advantage of the present device is that it can used for Sonophoresis (or phonophoresis). This is a technique in which therapeutic ultrasound energy, at certain exposure conditions, is used to increase the absorption of semisolid topical compounds and/or macromolecules through the skin (epidermis, dermis and skin appendages). The main biophysical mechanisms of action of sonophoresis are: (1) increasing the overall kinetic energy of molecules making up topical agents through ultrasound-induced radiation force, and (2) increasing the overall epidermis permeability through ultrasound-induced micro-vibrations and mild heating. The technique is generally used by mixing the topical compounds and/or macromolecules with an ultrasound coupling agent in a form of a gel, a cream, or an ointment. The present device is very effective for such application.

Sonophoresis for therapeutic applications including, but not limited, to enhancement of therapeutic oils and creams for pain and rejuvenation reasons using different therapeutic oils and creams including, but not limited, to cannabis CBD oils and creams.

The invention subject to this patent application possesses required technical features to allow it to be used in sonophoresis operations. This is due to the fact that the invention is capable of operating at output exposure parameters required for sonophoresis in terms of acoustic output power, and a wide range of output pulse sequencing (pulse width and pulse repetition frequency).

A variety of methods are used to restraining the vibrations for example providing an elastic on the upper electrode or provide a weight on the center of the upper electrode therefore restraining the undesired parasitic resonance on the applicator head.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1) A portable and handheld ultrasonic device for therapeutic purposes, comprising:
a) a piezo-crystal to generate an ultrasound vibration;
b) a stepwise signal driver to drive said piezo-crystal, wherein said stepwise signal driver increases the efficiency of said device by reducing the power loss;
c) a processor to control said stepwise signal driver, wherein said processor determines an operating resonance frequency of said piezo-crystal during an operation and under a loading condition to control said stepwise signal driver;
d) wherein said operating resonance frequency of said piezo-crystal is determined by maximizing a current passing through said piezo-crystal and by monitoring the change in shape and pattern of a voltage drop across a resistor in series with a bridge circuit, wherein an input frequency is adjusted at a predefined increment and the change in voltage pattern is measured until a maximum current is found;
e) a variable voltage source;
f) a power source, and
g) a user interface unit,
whereby said stepwise signal driver provides enough power to operate said ultrasonic device with a battery for a true handheld and portable operation.

2) The portable and handheld ultrasonic device of claim 1, wherein said stepwise driver generates voltage pulses comprising of an amplitude, a pulse duration (a), and a no pulse time (b), wherein said signal is optimized for more efficient operation by measuring the feedback voltage across a resistor (R) in series with a circuit bridge to control the pulse duration (a) and zero pulse time (b) to maximize the current passing through resistor R.

3) The portable and handheld ultrasonic device of claim 1, wherein said stepwise signal driver provides a nanosecond pulse pattern to drive said piezo-crystal.

4) The portable and handheld ultrasonic device of claim 1, wherein said ultrasound vibration is in a range of 0.5 to 5 MHz.

5) The portable and handheld ultrasonic device of claim 1, wherein said ultrasound device generates a combination of acoustic intensities from 0 W/cm² to 2 W/cm² and with 80-85% efficiency.

6) The portable and handheld ultrasonic device of claim 1, further having a temperature sensor to measure the temperature of the piezo-crystal.

7) The portable and handheld ultrasonic device of claim 1, further having a replaceable head, wherein each said replaceable head being designed for a specific application.

8) The portable and handheld ultrasonic device of claim 1, said piezo-crystal is identified by a coded bits.

9) The portable and handheld ultrasonic device of claim 1, further having a detector means to detect if the power is not delivered to a tissue and to measure the power of a delivered signal to the piezo-crystal, and to report said power to the processor.

10) The portable and handheld ultrasonic device of claim 1, wherein said processor comprises of a microcontroller based system for user interface communication, and having an On/Off button, a power and duty cycle level indicator, a LED visual interface, a wireless communication interface, a high-frequency/high-resolution signal generation, a control power level indicator by controlling a step up switching power supply, an automatic control for the piezo frequency tuning process, and a piezo type and frequency recognition device.

11) The portable and handheld ultrasonic device of claim 1, wherein said input power source is a universal voltage adaptor to adapt 110 Vac-240 Vac to 12 VDC/2 A with medical device category or a Li-ion Battery Pack 12.6V/2.4 A.

12) The portable and handheld ultrasonic device of claim 1, wherein said variable voltage or the switching power supply is responsible to deliver the necessary voltage/power to the driver stage based on a request from said processor.

13) The portable and handheld ultrasonic device of claim 1, wherein said user interface comprises of a switch, a display, and a communication link between the device and an external application on a smart phones/computers/Cloud.

14) The portable and handheld ultrasonic device of claim 1, further having a wireless communication interface to wirelessly communicate with any external processor and computers.

15) The portable and handheld ultrasonic device of claim 1, wherein said device is programmable to program the power, pulse rate and time of operation, enabling physician to track the treatment process and gradually apply the amount of power needed.
